(19) United States
(12) Patent Application Publication
(10) Pub. No.: US 2003/0096309 A1
(43) Pub. Date: May 22, 2003

(54) Screening system for identifying drug-drug interactions and methods of use thereof

(76) Inventors: Alexis Borisy, Boston, MA (US); Daniel Grau, Cambridge, MA (US); Brent R. Stockwell, Boston, MA (US); Curtis Keith, Boston, MA (US)

Correspondence Address: CLARK & ELBING LLP, 101 FEDERAL STREET, BOSTON, MA, 02110, US

(21) Appl. No.: 10/223,882

(22) Filed: Aug. 20, 2002

Related U.S. Application Data
(60) Provisional application No. 60/315,884, filed on Aug. 29, 2001.

(51) Int. Cl. G01N 33/53; G01N 33/543

(52) U.S. Cl. 435/7.1; 436/518

(57) ABSTRACT

The invention features a method of screening for drug-drug interactions using combinational arrays. The method includes the steps of: (a) providing (i) a test drug; (ii) a drug library; and (iii) an assay, (b) contacting the test drug and at least some of the library drugs from the drug library in the assay under conditions that ensure that each test drug/library drug contacting is segregated from the others, (c) recording the result of the contacting of the test drug and the library drug in the assay, and (d) identifying combinations of drugs that produce a result in the assay that is different from the results produced by either drug of the combination by itself. According to the method, each of the identified combinations indicates an interaction between the test drug and the library drug.

### CROSS-REFERENCE TO RELATED APPLICATIONS

This application claims benefit from co-pending U.S. Provisional Application No. 60/315,884 (filed Aug. 29, 2001), hereby incorporated by reference.

### BACKGROUND OF THE INVENTION

The invention relates to the fields of drug development and disease treatment.

Many disease states are associated with a multitude of phenotypic changes. This has long been apparent in the clinic, but recent advances in genomics have confirmed this observation at the molecular level. Expression profiling of cancer cells, for example, has revealed hundreds of changes in gene expression caused by multiple somatic mutations. Furthermore, human cells and tissues have evolved homeostatic mechanisms such that they often contain redundant and self-buffering signaling systems. Natural signals causing a change in cellular state are often sent not to a single target, but to the correct combination of targets. Thus, modest changes in multiple variables can have a highly specific effect.

In contrast, for historical and technological reasons, the pharmaceutical, chemical, and biological communities have primarily focused on single, individual molecules that cause biologic effects. This historical paradigm has resulted in the identification of small organic molecules that affect specific proteins, providing valuable reagents for both biology and medicine. These molecules are also useful as probes of the biological function of proteins that may have therapeutic relevance and have been effective at elucidating signal transduction pathways by acting as chemical protein knockouts, thereby causing a loss of protein function. Additionally, due to the interaction of these small molecules with particular biological targets and their ability to affect specific biological functions, they may also serve as candidates for the development of therapeutics.

Because it is impossible to predict which small molecules will interact with a biological target, intense efforts have been directed toward the generation of large numbers of small organic molecules. These are then gathered into what are called “libraries” of such compounds, with the goal of building a “diverse library” with the appropriate desired characteristics. Such a diverse library may be built from a pre-existing collection of small molecules or may be generated using “combinatorial chemistry.” These libraries can be linked to sensitive screens to identify active molecules (Stockwell et al. Chem. Biol. 1999, 6, 71-83).

In many cases, researchers have developed biased libraries, in which all members share a particular characteristic, such as an ability to interact with a target protein, or a characteristic structural feature designed to mimic a particular aspect of a class of natural compounds. For example, a number of libraries have been designed to mimic one or more features of natural peptides. Such “peptidomimetic” libraries include phthalimido libraries (WO 97/22594), thiophene libraries (WO 97/40034), and benzodiazepine libraries (U.S. Pat. No. 5,288,514). One library that has structural features reminiscent of natural products and that is compatible with miniaturized cell-based assays has been synthesized (Tan et al. J. Am. Chem. Soc. 1998, 120, 8565).

The modern drug discovery process is largely built upon an ability to assay rapidly compounds for their effects on biological processes. In the pharmaceutical industry, attention has been focused on identifying compounds that block, reduce, or even enhance the interactions between biological molecules. Specifically, in biological systems, the interaction between a receptor and its protein ligand often may result, either directly or through some downstream event, in either a deleterious or beneficial effect on that system, and consequently, on a patient for whom treatment is sought. Accordingly, researchers have long sought compounds that reduce, block, or even enhance such receptor/ligand interactions.

High throughput screening systems were designed to overcome the practical limitations on throughput for existing biochemical and cell-based assays. Traditional syntheses of organic compounds and traditional biological assays require significant time, labor, and skill. Screening the maximum number of compounds necessitates reducing the time and labor requirements associated with each screen.

High throughput screening of diverse collections of molecules has thus played a central role in the search for lead compounds for the development of new pharmacological agents. The inputs to these high throughput screens are libraries of compounds that have been assembled from preexisting chemically synthesized molecules (such as from a pharmaceutical company's proprietary library), natural products (such as microbial fermentation broths), and from novel libraries generated by combinatorial chemistry techniques (Tan et al. J. Am. Chem. Soc. 1998, 120, 8565). The libraries consist of up to a million compounds, which increases the likelihood of finding one compound with desirable properties to serve as a lead drug candidate (Tan et al J. Am. Chem. Soc. 1999, 121, 9073-9087).

Enhancing the traditional paradigm of drug discovery, combinatorial chemistry has resulted in a dramatic increase in the number of compounds that are available for screening, and human genome research has uncovered large numbers of new molecular targets for screening. Screens may use these new targets in a variety of ways, searching for enzyme inhibitors, receptor agonists or antagonists. The traditional goal is to find compounds that reduce, block, or enhance a single crucial interaction in a biological system (Weber et al., Angew. Chem. Int. Ed. Engl., 1995, 34, 2280-2282). Additionally, a number of researchers are adapting phenotype-based assay systems, where the screening is performed on whole, living cells, and the readout of the screen is some detectable property of the cell (Stockwell et al. Chem. Biol. 1999, 6, 71-83; Mayer et al. Science, 1999, 286, 971-974).

The high throughput screening methods developed to date have been designed based on the “one-drug-one-target” paradigm that dominates the pharmaceutical industry. For historical reasons, and because of regulatory considerations and perceived risk factors, the modern drug discovery process primarily looks to find one active molecule at a time to serve as a drug candidate. Clinicians have long recognized that the “one-drug-one-target” approach is not sufficient for the treatment of many diseases. They have tested some obvious combinations of agents that treat the same condition, or that have a clear logical connection. In HIV therapy and chemotherapy, for example, combinations of multiple active agents have become de rigeur. One of the most promising drugs of all times is a combination—Premarin, which is used to treat the complex changes in females after menopause, is composed of over 22 separate, active components.

In addition to possible beneficial interactions, it is also possible that, when administered together, two drugs may interact in an undesirable way. In one example, the activity of one or both drugs is generally increased, resulting in unacceptable side effects or a drug overdose. In another example, the presence of one drug counteracts or antagonizes the second drug, resulting in an insufficient amount of the second drug being administered. In still another example, two or more drugs interact to produce a non-additive amount of toxicity.

### SUMMARY OF THE INVENTION

We have invented methods for identifying drug-drug interactions. The methods involve high throughput screens of combinations of drugs to discover combinations that interact in biological assays. The methods of the invention can identify combinations of drugs that exhibit previously unknown effects even where each drug in the combination may have previously exhibited little or none of these effects.

Accordingly, in a first aspect, the invention features a method for identifying an interaction between two drugs. The method includes the steps of: (a) providing (i) a test drug; (ii) a drug library having at least 200 different drugs; and (iii) an assay, (b) contacting the test drug and at least 200 library drugs from the drug library in the assay under conditions that ensure that each test drug/library drug contacting is segregated from the others, (c) recording the result of the contacting of the test drug and the library drug in the assay, and (d) identifying combinations of drugs that produce a result in the assay that is different from the results produced by either drug of the combination by itself. According to the method, each of the identified combinations indicates an interaction between the test drug and the library drug.

In a second aspect, the invention features another method of determining whether a drug interacts with a member of a drug library. In this method, a client (e.g., a pharmaceutical company, a biotech company, an academic laboratory, or a governmental regulatory agency) provides a test drug (e.g., an FDA-approved drug or a drug being developed) to a service provider. The client, the service provider, or other entity provides a drug library having at least 200 different library drugs, and one or more assays. The service provider then (a) contacts the test drug and at least 200 library drugs from the drug library in the assay(s) under conditions that ensure that each test drug/library drug contacting is segregated from the others, (b) records the result of the contacting of the test drug and the library drug in the assay, and (c) identifies combinations of drugs that produce a result in at least one of the assays that is different from the results produced by either drug of the combination by itself. As in the first method, each of the identified combinations indicates an interaction between the test drug and the library drug.

In a third aspect, the invention features a method for screening two-drug or higher order combinations for biological activity. This method includes the steps of: (a) creating an array of at least 200 different two-drug or higher order combinations, (b) testing at least 200 of the combinations in an assay under conditions that ensure that each drug combination/assay contacting is segregated from the others, (c) recording the result of the testing of the combinations in the assay, and (d) identifying combinations of drugs that produce a result in the assay that is different from the results produced by either drug of the combination by itself, each of the identified combinations indicating an interaction between the test drug and the library drug of the combination.

In any of the first, second, or third aspects, the drug library can include drugs approved by the Food and Drug Administration (FDA) or other U.S. or non-U.S. governmental regulatory agency for administration to a human. The library can include, for example, 100, 200, 1000 approved drugs, or more. In one embodiment, all of the drugs in the drug library that are tested in one test are approved drugs. In another embodiment of any of the first, second, or third aspects, the method is repeated using at least three, five, ten, or more different assays.

In any of the foregoing aspects, the interaction may be one that is desirable. In one example, the interaction results in an increase in a desired biological activity without a concomitant increase in a second, undesirable biological activity. In another example, the interaction results in a reduction in an undesirable biological activity without reducing the amount of a second, desirable activity. In either of these examples, these drug combinations may be specifically co-prescribed for therapeutic purposes.

The interaction may also be one that is undesirable. For example, an interaction may reduce a desired activity of one or more of the drugs in combination. This reduction may be specific (i.e., the desired activity is reduced but undesired activities are maintained) or nonspecific (i.e., many or all activities are reduced). Interactions in this category may indicate proscription of an identified combination of drugs.

In a fourth aspect, the invention features a method for identifying an interaction between two drugs co-prescribed to a patient (e.g., a human or a non-human mammal such as a dog, cat, or horse). The method includes the steps of (a) providing (i) a test drug; (ii) a drug library that includes drugs that are co-prescribed (e.g., two drugs prescribed for the same disease, two drugs prescribed for two different diseases in the same patient, a drug to treat the disease and a second drug to treat a related co-morbidity, or a drug to treat a disease and a second drug to reduce a risk factor); and (iii) an assay, (b) contacting the test drug and at least some of the drugs in the library in the assay under conditions that ensure that each test drug/library drug contacting is segregated from the others, (c) recording the result of the contacting of the test drug and the library drug in the assay, and (d) identifying test drug/library drug combinations that produce a result in the assay that is different from the results produced by either drug of the combination by itself.

In the foregoing methods, the test drug can be a drug that is prescribed to patients having the disease for which the library drugs are also prescribed, or the test drug may be one that is prescribed to those patients once it has been approved by a governmental regulatory agency. The assay can be an assay known to detect activity of one or more of the drugs, or it can be an assay not known to detect activity of any of the drugs in the library.

An assay used in any of the first second, third, or fourth aspects can include one or more living human or non-human cells (e.g., cancer cells, immune cells, neurons, or fibroblasts) or can employ a cell-free system. One desirable assay is an assay that measures toxicity of the test drug/library drug combination. The recording step of the methods can employ, for example, a cytoblot assay, a reporter gene assay, a fluorescence resonance energy transfer assay, a fluorescent calcium-binding indicator dye, fluorescence microscopy, or gene expression profiling.

In the methods of the invention, each of the contacting step and the recording step can be performed manually or using a robotics system. The methods can also employ microfluidics and/or ink-jet printer technology. The drugs can be added to the assay in any sequence, i.e., one drug can be added to the assay, followed by the addition of a drug, or alternatively, the two drugs can be combined prior to their being contacted with the test element.

In another aspect, the invention features a library screening system for determining whether a test drug interacts with a member of a drug library in an assay. This screening system includes (i) a test drug; (ii) a drug library having at least 200 drugs; (iii) means for contacting the test drug and at least 200 of the drugs from the drug library in an assay under conditions that ensure that each test drug/library drug contacting is segregated from the others; (iv) a means for recording the result of the contacting; and (v) means for identifying test drug/library drug combinations that produce a result that is different from the result of either drug of the combination by itself.

As with the methods of the invention, the drug library of the screening system can include approved drugs. In one embodiment, the drug library includes at least 100, 200, or even 1000 approved drugs (e.g., drugs approved by the FDA). The recording means can employ, for example, a cytoblot assay, a reporter gene assay, a fluorescence resonance energy transfer assay, or a fluorescent calcium-binding indicator dye assay, utilizing a living cell or a cell-free system. The contacting means and/or the recording means can utilize a robotics system, microfluidics, and/or ink-jet printer technology.

Other features and advantages of the invention will be apparent from the following detailed description, and from the claims.

Definitions

“Drug”: As used herein, a “drug” is a substance used to treat or prevent a disease, or to ameliorate a manifestation of the disease, including but not limited to side effects and related risk factors and comorbidity. Also included in this definition are substances that are being developed for treatment or prevention of a disease, or amelioration of a manifestation of the disease. In many cases, drugs are small molecules. A “test drug” is a drug selected by the practitioner because the practitioner has a particular interest in the drug.

“Co-prescribed”: As used herein, “co-prescribed” refers to drugs that are often prescribed such that a person is taking the drugs concurrently. This term includes, for example, drugs that are prescribed to treat the same aspect of a condition (e.g., two anti-inflammatory agents), a drug to treat a condition and a drug to treat a side effect (e.g., a chemotherapeutic agent and an anti-emetic), a drug to treat a condition and a drug to aleviate pain, and two drugs prescribed to treat two different conditions that commonly occur in the same patient (e.g., osteoarthritis and type-2 diabetes), and a drug to treat a condition and a drug to treat a risk factor.

“Small molecule”: As used herein, “small molecule” refers to an organic drug either synthesized in the laboratory or found in nature, and that contains two or more carbon-carbon bonds, and has a molecular weight of less than 1500 g/mole.

“Assay”: As used herein, an “assay” is a process in which a combination of drugs is tested to determine one or more activities of the drugs.

“Drug printing”: As used herein, “drug printing” refers to the application of drugs to a surface (e.g., glass) using a high-precision robot such as that used in cDNA microarraying (J. Am. Chem. Soc. 1999, 121, 7967-7968). The drug spots can be 250 microns in diameter or smaller, and the drugs may be either covalently linked or adhering to the surface through electrostatic or hydrophobic interactions.

“Microfluidics”: As used herein, “microfluidics” devices are channeled structures made by any of the methods of photolithography, including conventional photolithography (e.g., Caliper Technologies, Mountain View, Calif.; http://www.calipertech.com) or unconventional methods (such as soft lithography, described, e.g., in Angew. Chem. Int. Ed. Engl. 1998, 37, 550-575).

“Ink-jet”: As used herein, “ink-jet” technology refers to both thermal ink-jet as well as piezoelectric spray technologies for delivery small volumes of liquids.

### BRIEF DESCRIPTION OF THE DRAWINGS

FIG. 1 is a conceptual diagram demonstrating how two different drugs could act synergistically inside of a cell, where the drugs bind to different targets within the same cell.

FIG. 2 is a conceptual diagram demonstrating how two different drugs could act synergistically inside of an organism, where the drugs bind to targets in different cells or tissues.

FIGS. 3A-3E show an example of the experimental data one would obtain in a combinatorial screen of the sort described herein. The results from five different 384 well plates are shown. The results are shown in plate format, where there are 16 rows labeled A through P, and 24 columns, labeled 1 through 24. The level of activity is shown in each well as a number, where 1 denotes basal activity (no effect) and 5 indicates an active combination is found.

FIG. 4 is a diagram of a method for performing combinatorial screening using currently commercially available technology.

FIGS. 5A and 5B are schematic illustrations showing the utilities of various interacting combinations, which vary depending on the type of interaction (enhancement or suppression) and the selectivity.

FIG. 6 is a schematic illustration showing results from interaction profiling of a drug (“A”) against 200 FDA-approved drugs (“1”-“200”), wherein the numbers represent the ratio of activity relative to control (in which nothing or placebo was added). In this illustration, the combination of drug A and drug 3 results in an enhancement of biological activity relative to the activity of either drug alone (combination: 7.5; drugs A and 3:2.2 and 1.2, respectively), while the combination of drug 6 (which has none of the desired activity) with drug A results in a suppression in drug A's biological activity (combination: 1.2; drug A alone: 2.2).

FIG. 7 is a schematic illustration showing results from interaction profiling of eight drugs commonly co-prescribed for treatment of osteoarthritis. The numbers represent the ration anti-inflammatory activity compared to control, as measured in an in vitro assay. In this example, only drugs 5 and 6 are prescribed for treatment of the inflammation per se, the other being given to treat or prevent side effects related to the disease, or a disease that commonly occurs in patients with osteoarthritis. Drug 3, which alone possesses little anti-inflammatory biological and is prescribed to treat type-2 diabetes, suppresses or antagonizes the activity of drug 6, but not drug 5. With this knowledge, a medical practitioner may elect to prescribe drug 5 instead of drug 6 to a patient suffering from both osteoarthritis and type-2 diabetes. New drug combinations may also be identified. For example, the combination of drugs 1 and 2 exhibits robust anti-inflammatory activity, although neither drug alone exhibits sufficient biological activity.

### DETAILED DESCRIPTION

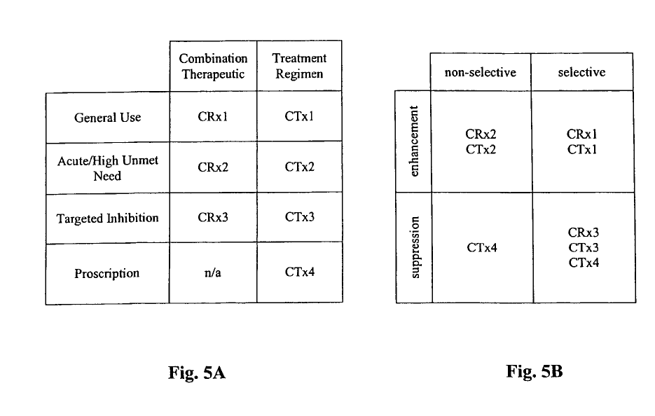

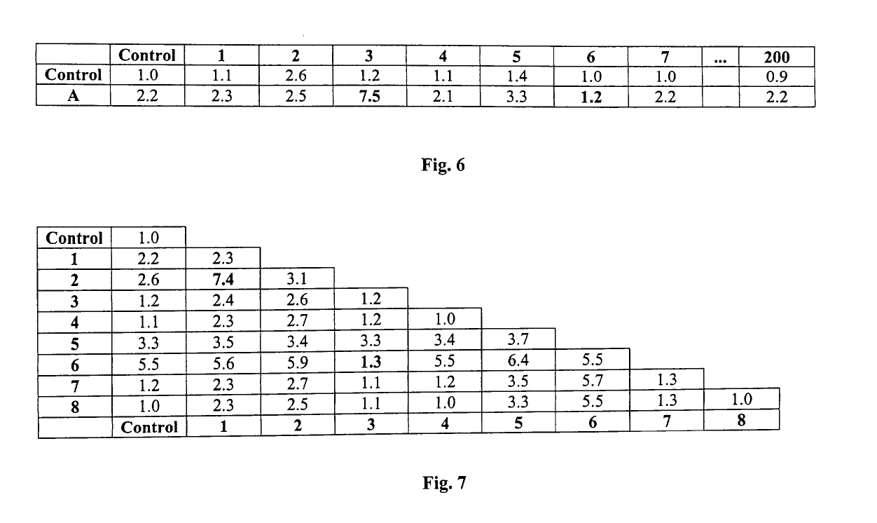

The present invention provides methods of interaction profiling to identify drug-drug interactions in a high throughput screening system using relevant biological assays to identify effects of the drugs that are only present in their unique combinations. Such combinations may have a direct biological use. In some instances, the combination displays a desirable activity that may serve as a human or animal therapeutic. In other instances, the combination results in a loss of desirable activity or an increase in an undesirable activity, uncovering important proscriptions and related regimens. Thus, the invention provides powerful methods for systematically performing high throughput screens of combinations of drugs to discover combinations having desirable or undesirable properties in biological systems (FIGS. 5A and 5B).

In some instances, the interaction between the drugs may be harmful, either due to increased or decreased activity of one or both drugs when administered in combination. Thus, the invention also provides methods for identifying these harmful interactions.

Drugs to be Tested in Combination

As is mentioned above, a wide variety of drugs can be tested in combination according to the invention. The prevalent class of drugs that are screened according to the invention are small organic molecules. The drugs can be either synthetic (e.g., recombinant oligonucleotides, proteins, or antibodies, organic or inorganic compounds, etc.) or naturally occurring (e.g., prostaglandins, lectins, naturally occurring secondary metabolites, hormones, etc.). Large libraries of such molecules, in purified form, are available in pharmaceutical companies, chemicals companies, and academic laboratories. In addition to small organic molecules, other drugs that can be screened in combination according to the invention include biopolymers, including DNA and RNA (e.g., antisense RNA or RNA used for RNA interference (RNAi)); polypeptides (antibodies, enzymes, receptors, ligands, structural proteins, mutant analogs of human proteins, and peptide hormones); lipids; carbohydrates; and polysaccharides.

It is desirable that the drug library include drugs approved by a governmental regulatory agency such as the FDA or EMEA. Other governmental regulatory agencies are listed below by country: Australia (Therapeutics Goods Administration), Austria (Federal Ministry of Labour, Health and Social Affairs), Belgium (Ministry of Public Health), Canada (Health Products and Food Branch), Denmark (Danish Medicines Agency), Finland (National Agency for Medicines), France (Agence du Médicament), Germany (BfArM/Paul-Erlich-Institut), Greece (National Drug Organisation), Iceland (The State Drug Inspectorate), Ireland (Irish Medicines Board), Israel (Ministry of Health), Italy (Ministry of Health), Japan (Ministry of Health and Welfare—Koseisho), Luxembourg (Division De La Farmacie Et des Médicamentes), Netherlands (Medicines Evaluation Board), New Zealand (Medicines and Medical Devices Safety Authority), Norway (Statens Legemiddelkontroll), People's Republic of China (State Drug Administration), Portugal (INFARMED—Instituto National da Farmácia e do Medicamento), Spain (Agencia Espaniola del Medicamento), Sweden (Medical Products Agency), and the United Kingdom (Medicines Control Agency).

Since interactions with other co-prescribed drugs may render a drug more, or less, commercially viable, a pharmaceutical company may wish to determine with which drugs a drug in development interacts. Accordingly, in one embodiment, to identify an interaction between two co-prescribed drugs, combinations of commonly co-prescribed drugs are screened in a method of the invention (FIG. 7). For example, Alzheimer's disease (AD) is commonly associated with anxiety, depression, aberrant motor activity, appetite changes, sleeping disturbances, delusions, and hallucinations. Thus a cholinesterase inhibitor (e.g., donepezil, tacrine, metrifonate, and rivastigmine), an antipsychotic drug (e.g., risperidone, clozapine), and a cholinomimetic drug may be co-adminstered to AD patients for the treatment of both the cognitive impairment and the related behavioral or psychological changes. In one scenario, the antipsychotic drug might specifically increases the cholinesterase activity of donepezil (with or without increasing side effects), indicating that donepezil should be given at lower dose when co-prescribed with the antipsychotic. In a second scenario, an antipsychotic agent might antagonize the cholinesterase inhibitor, indicating that the dosage of the cholinesterase should be increased. In another scenario, the side effects of one drug are increased in the presence of a second drug with out an appreciable increase in the desired drug activity. In still another scenario, the combination of two or more drugs exhibits an activity not present when each drug is administered alone. These latter two scenarios may indicate that two drugs should rarely or never be co-prescribed. Determining drug-drug interactions prior to extensive clinical testing can save a drug developer both time and money.

When screening co-prescribed drugs for drug-drug interactions, it is desirable to use both assays known to detect activity of one or both drugs, as well as assays not associated with either drug's known activity. In one example, because of the common association of osteoarthritis and type-2 diabetes, rosiglitazone and rofecoxib may be co-prescribed. To determine whether these two drugs are interacting, the drugs can be tested in an assay for antiinflammatory activity and an assay for rosiglitazone-mediated insulin utilization. Additionally, since the two drugs in combination may exhibit an activity not observed when either drug is tested alone, it is desirable also to test the combination in other assays, which measure effects unrelated to the indications for which the drugs are proscribed. For example, two or more drugs can be tested in three, five, ten, or more assays. Because of the ability to perform high throughput screening, performing twenty-five, fifty, or even one hundred different assays for 200 or more combinations is within the scope of the invention.

In one example of the type of information that can result from interaction profiling and how it could be used to provide better treatment, antiinflammatory drugs such as celecoxib and rofecoxib are each tested in combination with drugs commonly prescribed to treat or control type-2 diabetes in an assay that reports, for example, the antiinflammatory activity of the combination. The data could indicate that one of the drugs displays less antiinflammatory activity when in combination with, for example, rosiglitazone. At the clinical level, a practitioner could use this finding to devise an appropriate treatment regimen for patients having osteoarthritis and type-2 diabetes. At the regulatory level, a regulatory agency such as the FDA could use this information to decide whether a drug should be approved for a particular indication, if at all. At the drug discovery stage, a company could use the information in determining whether to choose a drug for clinical testing and FDA approval.

Once a drug-drug interaction has been identified, it may be desirable to determine whether structural or functional analogs of each drug in the combination also interact in the combination. For example, using the foregoing example of treatment of AD, should a harmful interaction between donepezil and clozapine be identified, one may wish to screen cholinesterase inhibitors and antipsychotic agents in all possible pair-wise combinations to determine which combinations have little or none of the harmful activity. Similarly, if Aricept and Prozac display a desirable activity in an assay for AD, this combination may be a new therapeutic option for treatment or management of AD.

Interaction Profiling

As is described herein, the invention provides methods for screening combinations of drugs for interactions in assays, a process referred to as “interaction profiling.” Interaction profiling allows for the construction of databases containing information regarding the interaction of two or more drugs in any number of assays. The information contained in these databases can be used for anything from rationale drug design to lead compound selection to determination of therapeutic combinations, and thus is valuable to the drug developer and clinical practitioner alike.

Library Screening System

The library screening system of the present invention includes a test drug, a drug library, and all of the components for performing the drug-drug interaction screening method, including the assay, means for performing the assay, and means for recording the results of the assay. Each of these latter components is described in greater detail below.

Assays

The biological assays used to detect the effects of the combinations will in most cases be composed of multiple components. Some assays include whole cells, particularly where the assay is a phenotype-based assay. Such a whole-cell assay provides the complete set of complex molecular interactions that are likely to form the basis of a drug's activity. Other assays employ a reconstituted cell free medium that contains many of the desired complex systems, and that may include some reporter effect that is based on the likely combinatorial effect being assayed for. Other assays employ higher order biological systems such as clusters of cells, tissues, and animal models.

Any biological assay that is useful for assay of individual drugs is readily adapted to the combinatorial screening of the present invention. Assay measurements can include, for example, toxicity, transport of a drug across the cell membrane, electrical potential, action potential generation, cell proliferation, cell death, cell specification, cell differentiation, cell migration, gene expression or protein levels (measured, e.g., by detecting mRNA, protein, or a reporter gene), enzymatic activity, phosphorylation, methylation, acetylation, translocation of a protein to the cell nucleus (or other change in protein localization), ability to resist a pathogen (e.g., a virus or a bacterium), and ability to produce an immune response. In whole organism assays, animal behavior can serve as a reporter.

In one example, the assay is a non-destructive assay (e.g., a cell-based assay in which a measurement of the effect of a drug can be obtained without harming the cells). Such an assay allows assays to be performed on multiple concentrations of multiple combinations per well. For example, drug A is added at increasing concentrations to a well and a measurement is taken after each addition of drug. When a desired concentration of drug A is reached (determined based on a desired assay response or on known properties (e.g., toxicity, solubility) of the drug), drug B is added in increasing concentrations, with an assay measurement taken after each addition. This process can be iterated many times in a single well, allowing hundreds, thousands, or even millions of assays to be performed in a single plate.

Cell-free media containing complex biomolecules such as proteins, carbohydrates, and lipids are made by known methods, e.g., by lysing mammalian, frog, yeast, or bacterial cells to provide a whole cell lysate, or by purifying a specific fraction from such a cell lysate, by using a commercially available rabbit reticulocyte lysate (commonly used for performing in vitro transcription and/or translation reactions), or by harvesting the culture supernatant from mammalian, yeast, or bacterial cells without lysing the underlying cells.

Cytoblot Assay

One method of detecting activity is the cytoblot assay. In this method, cells are seeded into wells of an assay plate. The cells are preferably adherent cells so they attach and grow on the bottom of the well. The drugs are added using the methods described above. For example, the cytoblot can be performed to detect proliferation by measuring the incorporation of BrdU. In this example, the cells are incubated for a set period of time, (e.g., 4 to 72 hours). The medium is then aspirated using, for example, a robotic liquid transfer device or a sixteen or eight channel wand. The cells are fixed by the addition of 70% ethanol and phosphate-buffered saline (PBS) at 4° C. for 1 hour. The fixitive is removed and the cells are washed once with PBS. After the PBS wash, 2N HCl with 0.5% Tween 20 is added to each well for 20 minutes. The HCl is neutralized with a solution of Hank's balanced salt solution (HBSS) containing 10% by volume of 2N NaOH. This solution is removed, the cells are washed twice with HBSS and then once with PBS containing 0.5% bovine serum albumin (BSA) and 0.1% Tween 20. The wash solution is removed and anti-BrdU antibody is applied as 0.5 μg/mL mouse anti-BrdU antibody in PBS containing 0.5% bovine serum albumin (BSA) and 0.1% Tween 20. This antibody solution also contains a secondary antibody (at a dilution of 1:2000) that recognizes mouse Ig antibody (e.g., the mouse anti-BrdU antibody); this secondary antibody is conjugated to the enzyme horseradish peroxidase (HRP). After one hour of incubation, the antibody solution is removed and the cells are washed twice with PBS. After the second PBS wash, the HRP substrate (which contains luminol, hydrogen peroxide, and an enhancer such as para-iodophenol is added to each well. The amount of light in each well is then detected using either exposure to film (by placing a piece of film on top of the plate) or by reading the amount of luminescence from each well using a luminometer or luminescence plate reader using standard conditions (e.g., 0.3 seconds of exposure per well). The amount of light output from each well indicates the amount of DNA synthesis that occurred in that well. A combination of agents is identified when there is either increased or decreased light output compared to a control. For example, a combination that decreases light output would be decreasing the rate of DNA synthesis and, thus, may be effective in prohibiting or preventing the proliferation of cells. Alternatively, an increase in light output represents an increase in DNA synthesis. For example, one could use primary cells in, for example, a 200×1 drug array (in which the one fixed component blocked DNA synthesis and, thus, had a toxic effect on the cells). Using this array, one could screen for a second drug that exacerbated this toxic effect of the first drug. Depending on the sensitivity of various cell types, this information may allow for the development of anticancer agents (if selective for neoplastic cells) or indicate that a given two-drug combination may be harmful.

Other Methods of Detecting Activity

The foregoing cytoblot assay is readily adapted to the detection of antigens other than BrdU. Moreover, one can detect a variety of post-translational modifications within cells. For example, an antibody against the phosphorylated version of nucleolin or histone H3 is useful for detecting cells that are in M (mitosis) phase of the cell cycle. Combinations of drugs that cause an increase in phosphorylated nucleolin or histone H3 in the cytoblot assay would therefore be combinations that arrest cells in M phase. One could also use a cytoblot assay to detect decreases in the acetylation of, for example, histone H4 using an antibody that specifically recognizes acetylated histone H4. Drugs or combinations of drugs that cause a decrease in hyperacetylation of histone H4 could be neoplastic agents.

In the foregoing examples, the procedure may be altered in that the medium is removed and a fixative (70% ethanol or 4% formaldehyde in PBS or Tris-buffered saline) is added. The membrane of the cells is then permeabilized by removing the fixative and adding a permeabilization agent (e.g., Tween 20, triton X-100, or methanol). The membrane permeabilization agent is removed, the cells are then washed with PBS or Tris-buffered saline, and then the primary antibody is added. There is usually no acid denaturation step using these other cytoblot embodiments.

Reporter Gene Assays

Recording the results if an assay may be performed with the use of a reporter gene. This method provides the advantage that, once the reagents (e.g., a stably transfected cell line) are prepared, the assay is easy to perform and requires less time than, for example, the cytoblot assay. Reporter genes include a reporter element, encoding a polypeptide that is readily detected due to a calorimetric, fluorescent, luminescent, or enzymatic property, and an enhancer/promoter element, which confers specificity to the expression of the reporter gene. Reporter elements include, without limitation, luciferase, beta lactamase, green fluorescent protein, blue fluorescent protein, chloramphenicol acetyltransferase (CAT), beta galactosidase, and alkaline phosphatase. Enhancer/promoter elements include, for example, those responsive to NFAT, p53, TGF-beta, or any other signaling pathway or stimulus for which a responsive promoter/enhancer is known.

The reporter gene can be introduced into the cells using any of a number of techniques, including, without limitation, transfection, viral or retroviral infection, bolistic injection, and cellular uptake of naked DNA. One in the art will recognize that any method of introduction of the reporter gene into the cells to be assayed will be compatible with the screening methods described herein.

Once the cells with the reporter gene are available, they are seeded in assay plates (96 well, 384 well, etc) with a pipette, multichannel pipette, 384 well Multidrop platefiller (Labsystems, Franklin, Mass.), or other liquid handling device. Drugs are added to form combinations by one of several methods. After 4-72 hours, the medium is removed, the cells are washed twice with HBSS, a lysis buffer is added (see Stockwell et al., J. Amer. Chem. Soc. 1999, 121:10662-10663), ATP/luciferin added and luminescence is measured on a platereader or luminometer (e.g. LJL BioSystems Inc., Analyst AD, Sunnyvale, Calif.).

Fluorescence Resonance Energy Transfer Assays

In another example, fluorescence resonance energy transfer (FRET) is used to detect and measure the interaction of two proteins of interest. In this example, the first and second proteins are fused with green fluorescent protein (GFP) and blue fluorescent protein (BFP), respectively, using standard molecular biological methods. The DNA constructs encoding the two fusion proteins are co-expressed in mammalian cells, yeast, worms, or other cell or organism using transfection techniques described above or other comparable methods. Combinations of drugs are added. The plate is placed on a platereader and fluorescence is measured as follows. The donor fluorophore (i.e., BFP) is excited and the emission of the acceptor fluorophore (i.e., GFP) is measured. Increased proximity of the two proteins will result in an increase in emission of the acceptor fluorophore. Thus, a combination of drugs that causes the two proteins of interest to be near each other is identified by an increase in fluorescence of the acceptor fluorophore.

For example, expression vectors containing Smad2 and Smad4 are obtained. The cDNA for GFP is fused to the 5′ end of Smad2 and the cDNA for BFP is fused to the 5′ end of Smad4. These expression vectors are transfected into mammalian cells stably or transiently, cells are treated with combinations of drugs, and the plate is irradiated with light that excites BFP but not GFP. Fluorescence of GFP (e.g., 512 nm light) is measured and combinations of drugs that cause an increase in light emission at this wavelength are identified. Such combinations are causing Smad2 and Smad4 to localize near each other, and may be activating TGF-beta signaling, and therefore may be useful for treating cancer chemotherapy mucositis, and autoimmune diseases.

Fluorescent Calcium Indicator Dyes

Another readout of a change in a property of a test element utilizes fluorescent calcium indicator dyes, such as fluo-3 (Molecular Probes, Eugene Wash.; http://www.molecularprobes.com), fura-2, and indo-1. Fluo-3 is essentially nonfluorescent unless bound to Ca²⁺ and exhibits a quantum yield at saturating Ca²⁺ of ˜0.14. The intact acetoxymethyl ester derivative of fluo-3 AM (fluo-3 AM) is therefore also nonfluorescent. The green-fluorescent emission (˜525 nm) of Ca²⁺-bound fluo-3 is conventionally detected using optical filter sets designed for fluorescein (FITC). According to the manufacturer, fluo-3 exhibits an at least 100-fold Ca²⁺-dependent fluorescence enhancement.

Cells are seeded in wells, fluo-3 is added to the cells following the manufacturer's instructions, drugs are added, and fluorescence is measured using a plate reader. Combinations of drugs that result in an increase in fluorescence (but are not themselves fluorescent) are thus causing an increase in the concentration of intracellular calcium.

Fluorescence Microscopy

Another assay uses conventional fluorescence microscopy to detect a change in the level or localization of fluorescence in cells contacted with combinations of drugs. In one example, a stably transfected cell line expressing a GFP tagged Smad2 is used. Cells are seeded, drugs are added and incubated for one hour, and a fluorescence microscope with an automated stage is used to image the cells in each well. Combinations of drugs that cause a change in the localization of the tagged protein are identified. For example, combinations that cause GFP-Smad2 to translocate from outside of the nucleus to the interior of the nucleus can be identified in this manner. These combinations may be activating TGF-beta signaling and, thus, may be useful for treating cancer, autoimmune diseases, and mucositis.

Expression Profiling with cDNA Arrays

Another assay for the detection of drugs that, together, produce an alteration in an assay is expression profiling. In this example, cells are seeded, combinations of drugs are added, and the cells are incubated for 2-24 hours. PolyA RNA is harvested from each well using standard methods. The RNA is reverse transcribed into cDNA using standard methods, with the exception that a fluorescent dye (e.g., Cy3-dUTP) is incorporated during the reverse transcription. The Cy3-labeled cDNA is mixed with Cy5-labeled cDNA from untreated cells and hybridized to a DNA microarray, (e.g., a DNA microarray commercially available from Affymetrix, Santa Clara, Calif., or Incyte, Palo Alto, Calif. reviewed in Nature Genet. Suppl. Jan. 21, 1999 (hereby incorporated by reference)). The relative level of Cy3 and Cy5 fluorescence at each spot in the microarray indicates which there has been a change in the expression of each gene. The method is used to identify combinations that cause an undesired change in gene expression.

Whole Organisms

Another bioassay that is compatible with the screening assays described herein utilizes a whole animal. In one example, the nematode C. elegans is placed into individual wells (preferably with more than one nematode per well), and the activity of drugs is detected by detecting a change in a property of the organism. For example, the nematode can be engineered to express green fluorescent protein at a specific stage of the life cycle, or only during the dauer state. An automated microscopy system is used to image the nematodes in each well and measure green fluorescent protein, or detect morphological changes in the worms caused by particular combinations of drugs.

Another whole animal assay uses large animals, such as nude mice, that have tumors on or near the skin surface. The combinations of drugs can be mixtures in DMSO that are rubbed into the skin, penetrate the skin, and reach the tumor. Alternatively, the drugs can be administered intravenously, intramuscularly, or orally. Other whole-organism methods of detecting activity could include using small tadpoles derived from fertilized Xenopus oocytes that develop in defined medium, organotypic cultures (explants from mice or other animals) in which the organ can be cultured for a period of time in a defined medium, and eggs (fertilized or unfertilized) from a variety of animals. Another assay measures the tension of cardiac tissue or muscle tissue stretched between two springs; drug combinations that modulate contraction would result in increased or decreased in the tension on those springs.

Labeling of Drugs

Although some assays can be conducted without any of the combined entities being labeled, in some embodiments one or more of the combined entities is labeled so that the effect of the combination in the assay can be recorded. Any of a wide variety of known labels can be used, e.g., techniques that have been used widely in biochemistry for protein affinity chromatography using biotin-streptavidin interactions or hexahistidine tagged proteins (Janknect et al., Proc. Natl. Acad. Sci. USA 1991, 88:8972; Wilcheck et al Methods in Enzymology, Wilcheck, M; Bayer, E. A. Eds. Academic Press Inc. San Diego, 1990; pp. 123-129).

Combining of the Drugs

The methods of the invention can use existing robotics systems, 96-well, 384-well, 1536-well or other high density stock plates and 96-, 384-, or 1536-well or other high density assay plates, with which it is possible to screen up to 150,000 drugs or more per week. The automation of this technology can be adapted according to the invention to screen combinations of molecules. The methods of the invention may also use microfluidics systems made either by conventional photolithography or by unconventional methods (such as soft lithography or near-field optical lithography) to miniaturize the process. The methods of the invention may also use ink-jet printing or drug printing technologies. Additionally, the methods of the invention may use trained technician labor to achieve the same results and throughput as the robotics systems. The combinations of drugs may be made prior to contact with the test element, or they may be in situ in the presence of the test element. These plating methods are described in more detail below.

Manual Plating

Drugs may be plated (i.e., added to the cells to be tested) manually. In one example, purified chemical drugs are manually combined and tested in a 7×7 combinatorial array. The drugs, which in this case include seven drugs, are in a stock plate. The drugs are combined in an assay plate. The operator plates a first drug (or plurality of drugs) from a well of the stock plate in one row of the assay plate and then plates one column in the assay plate. This is repeated with a second well from the stock plate, only the plating in the assay plate is one row over and one column over from those into which the first drug was plated. This process is repeated until the full set of combinations has been plated.

Robotic Plating

There are numerous methods for adding drugs to wells to form combinatorial arrays, and one skilled in the art will recognize that the following examples are for illustrative purposes and are in no way limiting.

In one example of robotic plating, a robotic liquid transfer systems is used. Transfer systems are commercially available from, for example, Beckman Coulter (Fullerton, Calif.), Tecan (Research Triangle Park, N.C.) or Zymark (Hopkinton, Mass.). The robotic system plates specific volumes of the first set of drugs into each well in a given row, such that row 1 will have the same drug, row 2 will have the same drug, etc. Then, the liquid transfer device plates the same set of drugs along the columns such that each column will receive the same drug (although different columns will have different drugs). Transfer systems can be adapted for transferring small volumes (e.g., 1 nL).

Another method for effective transfer utilizes ink-jet printing technology (Gordon et al., J. Med. Chem. 1994, 13:1385-1401; Lemmo et al., Curr. Opin. Biotechnol. 1998, 9:615-617). Ink-jet printers draw from a plurality of vessels containing test drugs; each drug from a source well is printed out or injected onto the surface in each individual row and column for each drug. As describe above, the next drug is printed out onto the next row and the next column, and this is iterated until the entire grid is plated with combinations of drugs.

Yet another method for adding drugs to an assay plate utilizes a microarray spotter as developed by Patrick Brown at Stanford University for spotting DNA. This device uses an eight-quill pen printing head and eight linear quillheads that are dipped in a stock plate and printed along every row. Subsequently, either the plate is rotated ninety degrees or the printing head is rotated ninety degrees; the printhead then prints along the columns. One could vary the size of the print head using, for example, two, four or sixteen printhead for standard 384 well plates, and higher numbers for higher density plates.

Yet another method for adding drugs to an assay plate uses a commercially available instrument called the Hydra (Robbins Scientific, Sunnyvale, Calif.), which can be equipped with 384 separate syringes that are capable of dropping a known volume from a standard stock plate of drugs.

An alternate method for the plating of test drugs uses microfluidics systems such as those commercially available from Caliper Technologies (Mountain View, Calif.) and directly applying that system to creating an array that would create this combination. In this case, arrays of combinations at the microscale are created using capillary flow to distribute the drug solutions to the intersection points on a matrix.

Alternative Plating Methods

One in the art will recognize that any plate configuration can be adapted to the screening methods of the invention. For example, a 16×16 square plate would have 256 wells instead of the 384 wells in a 16×24 plate. This would allow one to adapt any liquid addition system in which liquid is only added along the rows or only along the columns because one could simply rotate the square plate ninety degrees and allow addition in the other direction. One would also incorporate into this design, a square plate holder that would have the dimensions or footprint of a standard 96-well or 384-well microtiter plate so that the adapted microsquare plate would fit within any existing plate liquid handling system.

Another method of providing the drugs or elements to be tested in combination is to provide them in solid form rather than liquid form, e.g., as small amounts of dry powder. Thus, two dry components (or one wet and one dry component) can be mixed and then the combination added to the cells in the medium (in which the combined entities are soluble). Solid drugs include beads from a combinatorial synthesis on which a different drug is added. For example, beads could be added with a bead picker. In one example, the beads are magnetic and added using a magnet.

In the high throughput assays of the robotics application of the invention, each well must be spatially addressable independently, and it is preferably possible to withdraw both large (up to 100 μL) and small (down to 1 nL) of each drug from a stock plate. An exemplary robotic platform for performing combinatorial screening assays of the invention is described below.

A two station robotic platform is created. The first station harbors a simple XYZ robotic arm with an attached pin transfer device such as is available through VWR (cat#62409-608). A stock plate and an assay plate enter the station and the robotic arm drops the pins into the stock plate and transfers these pins into the assay plate, thereby delivering 1-1000 nL, depending on pin size (most typically, 50 nL are delivered). Different pin devices allow transfer of different combinations of drugs, as described above in the example. The second station of the robot is a piezo electric dispenser, capable of withdrawing large volumes (up to 10 microliters) from a stock well of a single drug and then dispensing small volumes into each well of an assay plate. For example, the Ivek Digispense 2000 system has a resolution of 10 nL and should be sufficient for this purpose.

Library Size

For small drug libraries (<1000 drugs), all binary (up to one half million combinations) and tertiary combinations (up to several hundred million combinations) can be tested using the systems described herein.

The power of combinations quickly generates effective libraries to identify drug-drug interactions. A library of 200 drugs in pair-wise combinations generates a dataset of 19,900 combinations. A library of 300 drugs in 3 way combinations generates approximately 4.5 million distinct combinations of drugs.

Assay Plates

Three types of assay plates are used. The size of the pins in the pin transfer devices described above is adapted to accommodate each size assay plate.

1) Commercial 384 well plates (e.g., NalgeNunc white opaque polystyrene cell culture treated sterile plates with lid, cat#164610)

2) Commercial 1536 well plates (e.g., Greiner or Coming)

3) Custom prepared 1536 or 6144 well plates (made from polydimethysiloxane, Dow Corning) and delran molds, and Omni trays.

Software to Manage Drug Additions

Microsoft Visual Basic or programming language is used, using conventional programming techniques, to write software to operate the instruments described above. The software will permit the instrument to read the barcode of a stock plate or assay plate, track the location of plates on the assay deck, and transfer the appropriate volume of the correct drug into the correct assay well. Thus all combinations to be screened are determined or selected beforehand, and the instrument carries out the combination screening in an automated format, requiring only simple operator steps, such as placing specified plates onto the assay deck and removing specified plates from the assay deck to an incubator.

Barcode Reader and Printer

A barcode printer is used, using standard techniques, to generate a unique identification number for each plate, print the barcode on a label, and stamp the label on the assay or stock plate. Software records the identity of each drug in each well of each assay plate and stock plate. A barcode reader linked to the assay deck scans each plate as it enters and leaves the assay deck.

The following example is provided for illustrative purposes, and is not meant to be limiting in any way.

### EXAMPLE 1

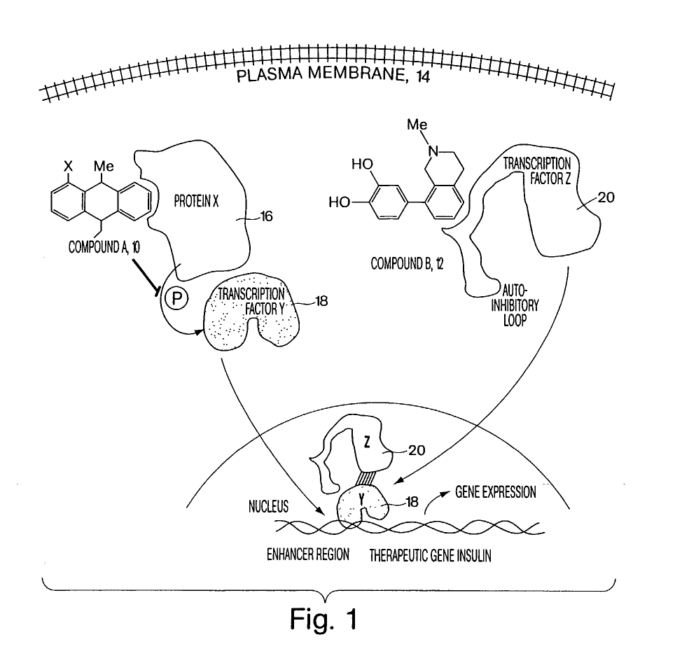

FIG. 1 is a conceptual diagram demonstrating how two different reagents could act synergistically inside of a cell, where the reagents bind to different targets within the same cell. In this figure, compound A 10 and compound B 12 cross the plasma membrane 14 and diffuse freely into the cytosolic region of a mammalian cell. Compound A binds to protein X 16, which is a kinase, inhibiting the activity of this kinase. Kinase X normally inactivates transcription factor Y 18 by adding a phosphate group to Y. Once compound A has inhibited kinase X, transcription factor Y is activated, and Y translocates into the nucleus, binding tightly to DNA in the enhancer region of a therapeutic gene, such as insulin. However transcription factor Y is unable to activate expression of insulin without the presence of a second transcription factor Z 20. However, in the figure, compound B binds to transcription factor Z, removing an autoinhibitory loop on this transcription factor, thereby causing this transcription factor Z to translocate into the nucleus, and bind to transcription factor Y. Y and Z together, but neither alone, allow activation of expression of the therapeutic gene, insulin.

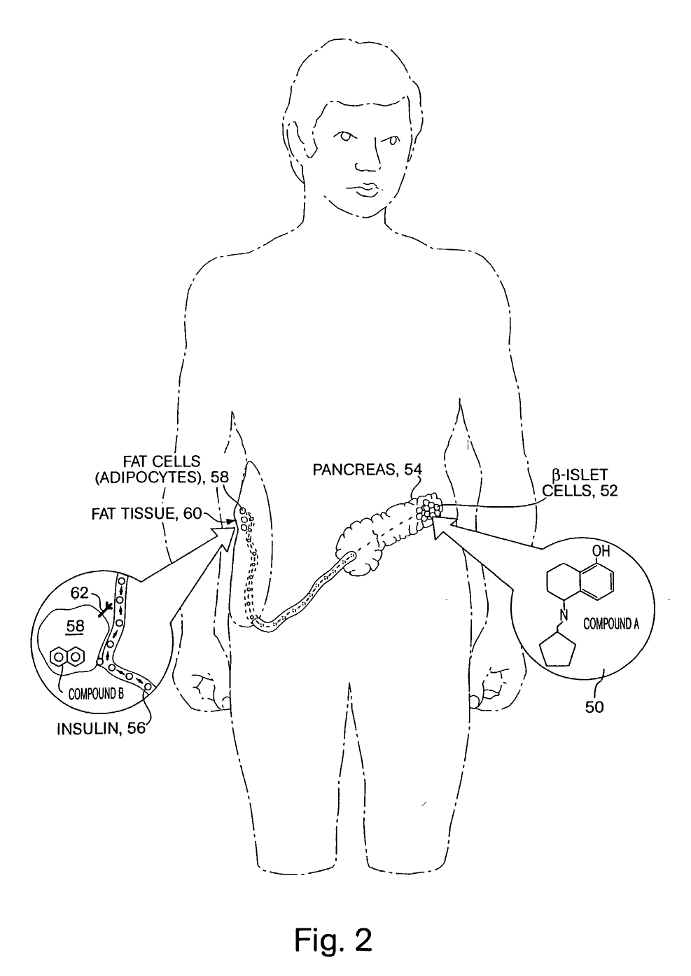

FIG. 2 is a conceptual diagram demonstrating how two different reagents could act synergistically inside of an organism, where the reagents bind to targets in different cells or tissues. In this figure compound A 50 diffuses into beta islet cells 52 of the pancreas 54. Compound A causes a therapeutic gene encoding insulin 56 to be expressed in these cells. However, insulin is ineffective without the presence of the insulin receptor on target adipocytes in fat tissue. Meanwhile, compound B diffuses into adipocytes 58 in fat tissue 60 and turns on expression of the insulin receptor 62 in these cells. A and B together, but neither one alone, allow insulin activity to be restored in this individual.

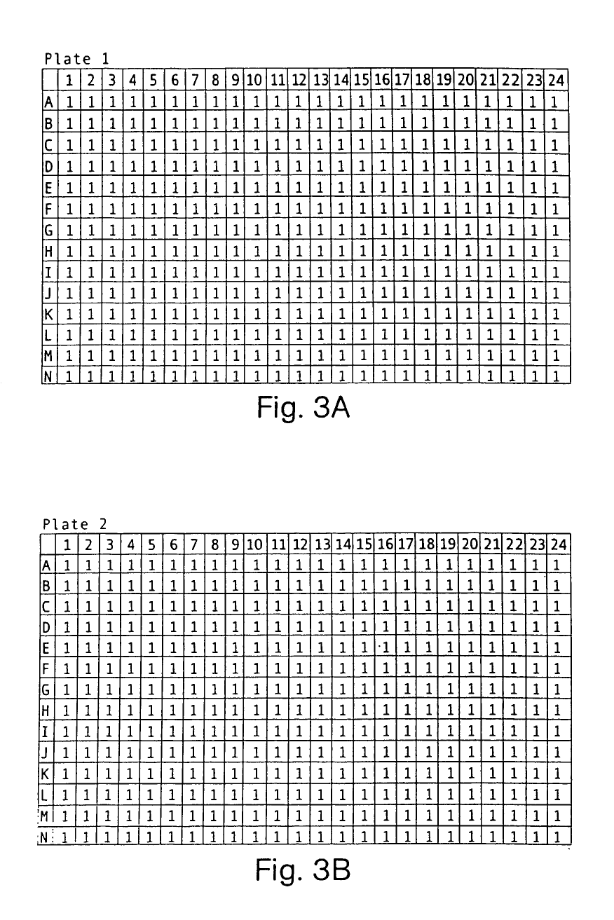

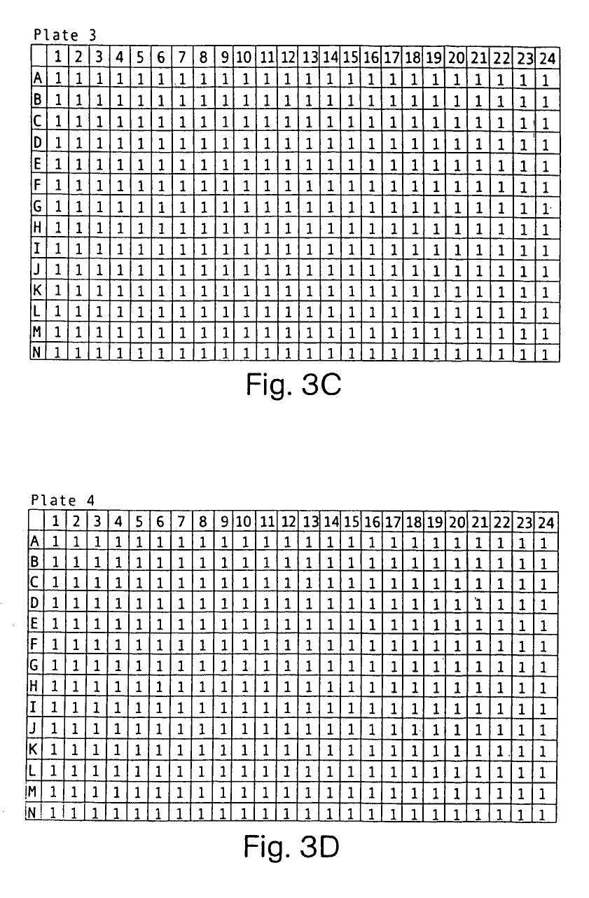

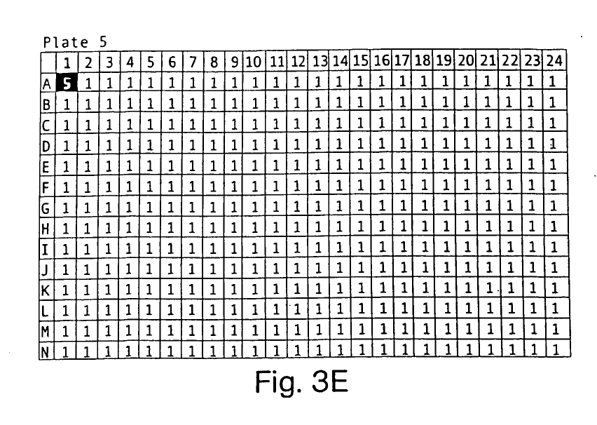

FIGS. 3A-3E show an example of the experimental data one would obtain in a combinatorial screen of the sort we describe in this patent application. The results from five different 384 well plates are shown. The results are shown in plate format, where there are 16 rows labeled A through P, and 24 columns, labeled 1 through 24. The level of activity is shown in each well as a number, where 1 indicates basal activity (no effect) and 5 indicates an active combination. Plate one shows the activity of compounds 1-384, when tested at 4 μg/mL in a bromodeoxyuridine cytoblot assay for cell-cycle arresting activity in A549 human carcinoma cells (described below). Plate two shows the activity of compounds 1-384 when tested at 2 μg/mL in same assay. Plate three shows the activity of compounds 385-768, when tested at 4 μg/mL, in the same assay. Plate four shows the activity of 385-768, when tested at 2 μg/mL, in the same assay. Note that in plates 1-4, none of the compounds shows any activity. Plate five shows the activity of 384 pair-wise combinations of compounds 1-768, when tested at 2 μg/mL (i.e., compounds 1-384 and 385-768 were both added simultaneously to the assay plate at 2 μg/mL each compound, creating 384 different random pair-wise combinations). Note that well A1 shows activity. This means that compound 1 (in well A1 of the plate with compounds 1-384) by itself had no activity and compound 385 (in well A1 of the plate with compounds 385-768) by itself had no activity but together compound 1 and 385 synergize to create an active combination.

### EXAMPLE 2

General Methods

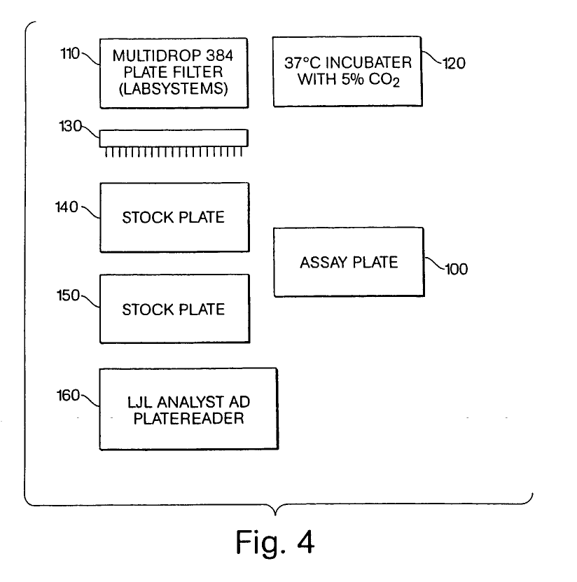

FIG. 4 is an illustration of a method for performing drug interaction screening using currently commercially available technology. Human cells are cultured in a suitable culture medium. Four thousand cells are seeded in each well of a white, opaque 384-well plate 100 (Nalge Nunc International, Rochester, N.Y.) using a Multidrop 384 liquid dispenser 110 (Labsystems Microplate Instrumentation Division, Franklin, Mass.). After 16 hours at 37 C with 5% CO₂, 10 μL of a 50 μM stock of a drug of interest in 10% medium is added to each well, bringing the total well volume to 40 μL and the final concentration of this drug to 10 μM. Either prior to, immediately afterwards, or several hours or days later, a set of drugs from a drug library is added by dipping small pins on a pin array 130 into each well of a stock plate 140 or 150 and then into each well of an assay plate 100. Matrix Technologies Pin Transfer device 130 (384 or 96 pins) will suffice (catalog numbers 350500130 and 350510203). This two-step process allows for the testing of one specific drug against a large number of other drugs in many pair-wise combinations. It is necessary to also have a plate where the set of pin-transferred drugs is tested in the absence of the original drug (the one present in all wells) to determine whether a novel property has been achieved with the combination.

A different method can also be used to provide combinations of drugs for in the assay. For example, instead of keeping one drug fixed throughout the pair-wise combinations, as described above, it is possible to pin transfer a set of drugs from a drug library in such a way that all pairwise combinations of that set are achieved. A set of 16 drugs is pin transferred from stock plate 140 to the 16 rows of the 384 well assay plate 100. The same set of 16 drugs is then transferred to 16 columns of the same assay plate, providing a 256 well matrix with different pairwise combinations (including duplicate wells and wells in which the same drug has been added twice).

### EXAMPLE 3

The identification of a combination of drugs that inhibit proliferation is described below.

Seven compounds were tested alone and in all 21 possible pair-wise combinations in the BrdU cytoblot assay (see below) for their effect on cell cycle progression. The seven compounds (podophyllotoxin, paclitaxel, quinacrine, bepridil, dipyridamole, promethazine, and agroclavine; each purchased from Sigma Aldrich Corp., St. Louis, Mo.) were weighed into one dram glass vials and dissolved in dimethylsulfoxide to create 4 mg/mL stock solutions. Six thousand A549 lung carcinoma cells were seeded in each well of a 384 white opaque NalgeNunc cell culture-treated plate (cat#164610) in 30 μL of 10% medium (Dulbecco's Modified Eagle Medium containing 10% fetal bovine serum, 100 units/mL penicillin G sodium, 100 μg/mL streptomycin sulfate, and 2 mM glutamine, all obtained from Life Technologies). Each compound was diluted to four times the final assay concentration (final assay concentrations were 0.25% DMSO, 240 nM podophyllotoxin, 60 nM paclitaxel, 420 nM quinacrine, 25 μM bepridil, 400 nM dipyridamole, 25 μM promethazine, 840 nM agroclavine) in 10% medium. Fifteen microliters of each 4×stock of compound in medium was added to one row and one column of an eight column and eight row square (the eighth lane containing only the vehicle DMSO), such that all possible binary combinations of the seven compounds were tested, as well as the single agents themselves. The cells were incubated at 37° C. with 5% carbon dioxide for 46 hours. BrdU was added to a final concentration of 10 μM by adding 15 μL of a 50 μM solution of BrdU in 10% medium. The cells were incubated overnight at 37° C. with 5% carbon dioxide.

After 16 hours the medium was aspirated from each well with a 24-channel wand (V&P Scientific), used throughout the protocol, attached to a house vacuum source. Fifty microliters of 70% ethanol/30% phosphate buffered saline (4° C.) was added to each well with a Multidrop 384 plate filler (Labsystems), used for all subsequent liquid addition steps. The plate was incubated for one hour at room temperature, then the wells were aspirated, and 25 μL of 2 M HCl with 0.5% Tween 20 was added to each well. The plate was incubated for 20 minutes at room temperature. Twenty five microliters of 2 M NaOH was then added to each well. The liquid in each well was aspirated and the wells were washed twice with 75 μL of Hank's Balanced Salt Solution. The wells were washed again with 75 μL of PBSTB (phosphate buffered saline with 0.5% bovine serum albumin and 0.1% Tween 20). Twenty microliters of antibody solution was added to each well (containing 0.5 μg/mL anti-BrdU antibody (PharMingen) and 1:2000 dilution of anti-mouse Ig-HRP (Amersham). The plate was incubated with the antibody solution for one hour at room temperature, then the antibody solution was aspirated off and each well was washed once with phosphate buffered saline. Finally, 20 μL of ECL detection reagent was added to each well (an equal mixture of solutions one and two from Amersham's ECL detection reagents). The luminescence in each well was read on an LJL Analyst platereader with 0.2 seconds integration time per well. The experiment was repeated in two plates and each pair-wise combination was tested in a total of sixteen replicate experiments. The data shown in Table 1 depict the mean antiproliferative activity of each combination of compounds. Five statistically significant (p<0.001) combinations are highlighted. All activity is normalized to a set of wells containing cells that did not receive any treatment. Thus, a value of one represents an inactive substance and a value greater than one indicates some level of antiproliferative activity

TABLE 1

| | DMSO | Podophyllotoxin | Paclitaxel | Quinacrine | Bepridil | Dipyridamole | Promethazine | Agroclavine |
|---|---|---|---|---|---|---|---|---|
| DMSO | 1.0 | | | | | | | |
| Podophyllotoxin | 6.5 | 6.5 | | | | | | |
| Paclitaxel | 6.3 | 6.3 | 7.7 | | | | | |
| Quinacrine | 1.7 | 6.6 | 6.9 | 2.3 | | | | |
| Bepridil | 1.8 | 15.2 | 3.1 | 3.0 | 14.4 | | | |
| Dipyridamole | 1.5 | 8.9 | 8.8 | 2.3 | 2.4 | 2.0 | | |
| Promethazine | 1.3 | 3.9 | 6.4 | 2.4 | 15.2 | 1.8 | 4.8 | |
| Agroclavine | 1.0 | 5.7 | 5.8 | 1.6 | 1.9 | 1.5 | 1.2 | 1.0 |

The chart shows five combinations of existing FDA-approved drugs with antiproliferative activity that is distinct from that of the individual components. Podophyllotoxin and paclitaxel are both microtubule stabilizers that arrest cells in mitosis, dipyridamole is an anti-platelet agent, bepridil is a calcium channel blocker, and promethazine is an H1 histamine receptor antagonist and is also used as a CNS depressant and anticholinergic agent. Dipyridamole is generally considered to have a relatively high safety profile as a human therapeutic, particularly compared to the toxic side effects of paclitaxel and podophyllotoxin. Thus, in this assay, dipyridamole enhances the antiproliferative effect of both paclitaxel and podophyllotoxin on human lung cancer cells. Furthermore, bepridil enhances the effects of podophyllotoxin but inhibits the effect of paclitaxel. This result would not have been predicted a priori and highlights the importance of empirical high throughput testing of combinations to observe unexpected interactions among drugs. For example, bepridil and promethazine, neither of which is used as an antiproliferative agent in current therapeutic indications, combine to strongly inhibit the proliferation of lung cancer cells.

### EXAMPLE 4

The identification of a combination of drugs that inhibit TNFα secretion is described below.

Stock solutions of amoxapine (16 mg/ml) (Sigma-Aldrich, St. Louis, Mo.; catalog number A129) and prednisolone (1.6 mg/ml) (Sigma-Aldrich, catalog number P6004) were made in dimethylsulfoxide (DMSO). The amoxapine master plates were made by adding 25 μl of the concentrated stock solution to columns 3, 9, and 15 (rows C through N) of a polypropylene 384-well storage plate that had been pre-filled with 75 μl of anhydrous DMSO. Using a TomTec Quadra Plus liquid handler, the 25 μl of amoxapine stock solution was serially diluted two-fold four times into the adjacent columns (columns 4-7, 10-13, 16-19). The sixth column (8, 14, and 20) did not receive any compound and served as a vehicle control. The prednisolone master plates were made by adding 25 μl of the concentrated prednisolone stock solution to the appropriate wells (row C, columns 3-8; row C, columns 9-14; row C, columns 15-20; row I, columns 3-8; row I, columns 9-14; row I, columns 15-20) of the appropriate prednisolone master polypropylene 384-well storage plate. These master plates had been pre-filled with 75 μl of anhydrous DMSO. Using the TomTec Quadra Plus liquid handler, the 25 μl was serially diluted two-fold four times in the adjacent rows (rows D-G, and J-M). The sixth row (H and N) did not receive any compound to serve as a vehicle control. Master plates were sealed and stored at −20 C until ready for use.

The final amoxapine/prednisolone combination plate was generated by transferring 1 μl from each of the amoxapine and prednisolone master plates to a dilution plate containing 100 μl of media (RPMI; Gibco BRL, #11875-085), 10% Fetal Bovine Serum (Gibco BRL, #25140-097), 2% Penicillin/Streptomycin (Gibco BRL, #15140-122)) using the TomTec Quadra Plus liquid handler. This dilution plate was then mixed and a 10 μl aliquot transferred to the final assay plate, which had been pre-filled with 40 μl/well RPMI media containing the appropriate stimulant to activate TNFα secretion (see below).

The compound dilution matrix was assayed using a TNFα ELISA method. Briefly, a 100 μl suspension of diluted human peripheral blood mononuclear cells (PBMCs) contained within each well of a polystyrene 384-well plate (NalgeNunc) was stimulated to secrete TNFα by treatment with a final concentration of 10 ng/ml phorbol 12-myristate 13-acetate (Sigma) and 750 ng/μl ionomycin (Sigma). Various concentrations of each test compound were added at the time of stimulation. After 16-18 hours of incubation at 37° C. in a humidified incubator, the plate was centrifuged and the supernatant transferred to a white opaque polystyrene 384 well plate (NalgeNunc, Maxisorb) coated with an anti-TNF antibody (PharMingen, #18631D). After a two-hour incubation, the plate was washed (Tecan PowerWasher 384) with phosphate buffered saline (PBS) containing 0.1% Tween 20 (polyoxyethylene sorbitan monolaurate) and incubated for an additional one hour with another anti-TNF antibody that was biotin labeled (PharMingen, 18642D) and horseradish peroxidase (HRP) coupled to strepavidin (PharMingen, #13047E). After the plate was washed with 0.1% Tween 20/PBS, the HRP substrate (which contains luminol, hydrogen peroxide, and an enhancer such as para-iodophenol) was added to each well and light intensity measured using a LJL Analyst luminometer. Control wells contained a final concentration of 1 μg/ml cyclosporin A (Sigma).

Together, amoxapine and prednisolone were able to suppress phorbol 12-myristate 13-acetate and ionomycin induced TNFα secretion in blood. As is shown in Tables 2 and 3, amoxapine is able to enhance the dose response of prednisolone by nearly two fold. At a concentration of 1.11 μM, prednisolone alone is able to inhibit TNFα secretion by 28%. Addition of 0.2 μM amoxapine increases the TNFα inhibition of the 1.11 μM prednisolone to 51%. This large increase in activity of 82% is created by a relatively small increase of only 18% in total drug species.

TABLE 2

| | | Amoxapine [μM] | | | | | |
|---|---|---|---|---|---|---|---|
| | | 12.750 | 3.188 | 0.797 | 0.199 | 0.050 | 0.000 |
| Prednisolone | 1.110 | 85.89 | 66.47 | 47.73 | 50.93 | 32.79 | 27.59 |
| [μM] | 0.277 | 82.82 | 58.88 | 48.79 | 34.40 | 29.17 | 26.81 |
| | 0.069 | 78.58 | 60.66 | 41.32 | 34.99 | 21.41 | 22.31 |
| | 0.017 | 84.69 | 62.66 | 34.66 | 32.48 | 21.39 | 19.06 |
| | 0.004 | 84.13 | 53.41 | 33.86 | 16.02 | 5.82 | 2.69 |
| | 0.000 | 73.02 | 50.44 | 24.29 | 16.66 | 7.30 | 0.00 |

TABLE 3

| | | Amoxapine [μM] | | | | | | | | | |
|---|---|---|---|---|---|---|---|---|---|---|---|
| | | 12.747 | 6.373 | 3.187 | 1.593 | 0.797 | 0.398 | 0.199 | 0.100 | 0.050 | 0.000 |
| Prednisilone | 1.110 | 88.35 | 74.63 | 66.76 | 65.79 | 57.72 | 52.50 | 45.50 | 43.72 | 40.04 | 34.62 |
| [μM] | 0.555 | 90.09 | 76.42 | 69.66 | 60.08 | 53.51 | 46.73 | 41.70 | 43.67 | 31.76 | 30.50 |
| | 0.277 | 86.67 | 75.34 | 66.45 | 59.64 | 54.23 | 46.97 | 45.38 | 35.20 | 34.42 | 36.89 |
| | 0.139 | 91.50 | 78.45 | 70.37 | 60.75 | 54.73 | 47.05 | 41.51 | 37.79 | 28.46 | 25.74 |
| | 0.069 | 36.59 | 86.03 | 77.74 | 67.81 | 57.14 | 49.96 | 37.24 | 33.39 | 31.75 | 24.56 |
| | 0.035 | 92.76 | 80.28 | 70.42 | 59.40 | 52.58 | 47.40 | 37.94 | 34.59 | 21.47 | 24.06 |
| | 0.017 | 91.02 | 75.16 | 72.06 | 56.40 | 45.14 | 47.84 | 36.50 | 24.33 | 21.92 | 24.74 |
| | 0.009 | 88.58 | 72.16 | 71.61 | 56.03 | 49.80 | 39.87 | 28.66 | 27.23 | 17.78 | 14.34 |
| | 0.004 | 84.32 | 66.14 | 57.21 | 46.53 | 32.06 | 27.76 | 23.73 | 15.94 | 12.99 | 9.62 |
| | 0.000 | 80.89 | 64.40 | 47.96 | 37.13 | 21.88 | 16.38 | 14.19 | 3.60 | −3.31 | −0.97 |

Amoxapine enhancement of prednisolone activity was also observed in a follow-up secondary screen. The TNFα inhibition of prednisolone at a concentration of 9 nM was increased 2.9 fold to 40% in the presence of 400 nM amoxapine. The TNFα inhibition activity of prednisolone and amoxapine alone at these concentrations is only 14 and 16% respectively. Moreover, the level of TNFα inhibition achieved by 9 nM prednisolone in combination with 398 nM amoxapine (40%) is no less than that of 1110 nM prednisolone alone (35%). This increase in TNFα inhibition constitutes a potency shift of as much as 100-fold for the combination, compared to prednisolone alone.

The ability of amoxapine and prednisolone to inhibit TNFα secretion from LPS stimulated blood is shown in Table 4.

TABLE 4

| | | Amoxapine [μM] | | | | | | | | | |
|---|---|---|---|---|---|---|---|---|---|---|---|
| | | 12.747 | 6.373 | 3.187 | 1.593 | 0.797 | 0.398 | 0.199 | 0.100 | 0.050 | 0.000 |
| Prednisolone | 1.110 | 78.97 | 71.52 | 67.84 | 63.65 | 66.04 | 68.04 | 61.29 | 64.30 | 58.19 | 60.06 |
| [μM] | 0.555 | 83.61 | 68.05 | 62.72 | 65.82 | 59.46 | 56.17 | 56.36 | 55.47 | 55.94 | 47.15 |
| | 0.277 | 70.40 | 64.01 | 62.08 | 57.91 | 55.42 | 54.64 | 56.94 | 51.39 | 50.05 | 48.75 |
| | 0.139 | 72.56 | 60.77 | 58.36 | 55.47 | 50.42 | 49.25 | 49.54 | 48.74 | 44.46 | 48.46 |
| | 0.069 | 70.27 | 73.99 | 61.88 | 48.82 | 43.56 | 47.22 | 42.13 | 42.62 | 44.19 | 38.79 |
| | 0.035 | 86.37 | 64.17 | 43.28 | 38.16 | 37.26 | 37.96 | 38.06 | 40.83 | 32.87 | 33.11 |
| | 0.017 | 78.81 | 48.94 | 42.94 | 40.81 | 37.94 | 35.96 | 32.00 | 35.25 | 29.35 | 37.12 |
| | 0.009 | 67.09 | 43.76 | 41.07 | 34.23 | 25.54 | 24.86 | 31.12 | 23.57 | 27.36 | 30.24 |
| | 0.004 | 60.14 | 37.59 | 34.03 | 25.52 | 24.94 | 27.78 | 25.57 | 30.40 | 18.90 | 12.06 |
| | 0.000 | 49.64 | 21.26 | 24.21 | 16.79 | 13.11 | 8.10 | 2.39 | 5.52 | 3.00 | −1.31 |

### EXAMPLE 5

The identification of a combination of drugs in which one drug antagonizes the effect of a second drug is described below.

The combination of dexamethasone and econazole provides an interesting example of drugs whose therapeutic interaction is dictated by the experimental or clinical context in which it is applied. In the case of PMA-Ionomycin stimulation (which primarily activates the T-cell arm of the immune system, these two drugs interact synergistically; 45% inhibition of TNF-alpha secretion is achieved at a dexamethasone dose of 4 nM in the presence of 281 nM econazole, whereas in the absence of econazole, this level of TNA-alpha suppression is not achieved until the dexamethasone dose reaches 32 nM. The potency of the steroid dexamethasone is therefore increased at least 8-fold by the presence of the anti-fungal drug econazole (Table 5).

TABLE 5

| | | Dexamethasone [μM] | | | | | | | | | |
|---|---|---|---|---|---|---|---|---|---|---|---|
| | | 0.255 | 0.127 | 0.064 | 0.032 | 0.016 | 0.008 | 0.004 | 0.002 | 0.001 | 0.000 |
| Econazole | 8.995 | 72.94 | 74.84 | 69.98 | 67.65 | 69.51 | 59.60 | 62.47 | 57.60 | 60.22 | 54.10 |
| [μM] | 4.497 | 60.00 | 55.25 | 53.69 | 56.81 | 50.38 | 45.66 | 40.01 | 41.17 | 38.09 | 19.08 |
| | 2.249 | 57.25 | 53.21 | 54.75 | 56.71 | 54.42 | 44.13 | 42.23 | 37.52 | 37.71 | 22.36 |
| | 1.124 | 54.88 | 55.47 | 53.81 | 46.54 | 41.64 | 44.85 | 45.01 | 41.06 | 29.84 | 14.29 |
| | 0.562 | 47.83 | 49.00 | 49.26 | 53.14 | 47.89 | 43.32 | 41.45 | 39.23 | 31.57 | 10.54 |
| | 0.281 | 52.22 | 51.65 | 48.85 | 48.23 | 50.51 | 46.85 | 45.06 | 36.99 | 28.60 | 12.82 |
| | 0.141 | 46.59 | 51.39 | 44.30 | 42.00 | 37.92 | 38.10 | 35.59 | 31.09 | 19.29 | 4.04 |
| | 0.070 | 45.94 | 46.54 | 49.15 | 46.22 | 41.92 | 36.57 | 30.96 | 27.83 | 23.09 | 0.31 |
| | 0.035 | 47.35 | 49.10 | 47.73 | 46.58 | 40.19 | 39.21 | 34.53 | 30.62 | 21.78 | 3.21 |
| | 0.000 | 46.82 | 48.79 | 48.04 | 44.36 | 40.43 | 39.47 | 33.27 | 29.19 | 21.45 | 7.31 |

In the case of lipopolysaccharide stimulation (which activates primarily the macrophages), the interaction of dexamethasone and econazole is antagonistic at the level of TNF-alpha secretion. Dexamethasone alone is a more potent inhibitor of the macrophages inhibiting TNF-alpha secretion by 40% at a dose of 4 nM. In the presence of high doses of econazole, the TNF-alpha suppression is antagonized, and greater doses of dexamethasone are required to achieve the same level of activity. For example, at an econazole dose of 9 μM, 127 nM dexamethasone is required to achieve the same 40% inhibition of TNF-alpha representing an antagonistic effect of greater than 30-fold (Table 6).

TABLE 6

| | | Dexamethasone [μM] | | | | | | | | | |
|---|---|---|---|---|---|---|---|---|---|---|---|
| | | 0.255 | 0.127 | 0.064 | 0.032 | 0.016 | 0.008 | 0.004 | 0.002 | 0.001 | 0.000 |
| Econazole | 8.995 | 45.14 | 39.30 | 31.51 | 26.67 | 23.61 | 12.46 | −6.72 | −16.17 | −33.49 | −48.15 |
| [μM] | 4.497 | 55.83 | 49.91 | 52.36 | 46.24 | 43.18 | 37.30 | 27.63 | 12.58 | −5.88 | −25.41 |
| | 2.249 | 62.85 | 62.25 | 58.32 | 52.81 | 54.71 | 45.48 | 38.24 | 49.79 | 18.30 | −2.24 |
| | 1.124 | 66.55 | 60.61 | 61.55 | 59.20 | 55.11 | 50.71 | 46.43 | 39.37 | 31.10 | 2.12 |
| | 0.562 | 64.56 | 63.30 | 62.74 | 57.26 | 54.96 | 51.57 | 41.92 | 38.57 | 34.49 | 0.90 |
| | 0.281 | 64.87 | 62.02 | 59.70 | 57.47 | 59.23 | 54.88 | 48.86 | 41.94 | 29.52 | 4.51 |
| | 0.141 | 61.92 | 61.73 | 61.66 | 54.38 | 54.34 | 52.10 | 47.92 | 42.31 | 29.52 | 12.28 |
| | 0.070 | 59.81 | 60.26 | 58.64 | 56.51 | 53.96 | 51.12 | 48.04 | 34.87 | 33.92 | 6.90 |
| | 0.035 | 61.79 | 60.79 | 60.40 | 56.63 | 51.69 | 46.82 | 47.92 | 35.39 | 22.67 | 9.05 |
| | 0.000 | 58.59 | 56.01 | 54.96 | 54.23 | 47.87 | 40.88 | 39.50 | 28.15 | 22.48 | 0.32 |

### Other Embodiments

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the field of drug discovery or related fields are intended to be within the scope of the invention.

What is claimed is:

1. A method for identifying an interaction between two drugs, said method comprising the steps of:
(a) providing (i) a test drug; (ii) a drug library having at least 200 different drugs; and (iii) an assay;
(b) contacting said test drug and at least 200 library drugs from said drug library in said assay under conditions that ensure that each test drug/library drug contacting is segregated from the others;
(c) recording the result of said contacting step (b); and
(d) identifying combinations of drugs that produce a result in said assay that is different from the results produced by either drug of the combination by itself, wherein each identified combinations indicates an interaction between said test drug and said library drug of said combination.

2. The method of claim 1, wherein at least 200 of said library drugs of step (a) are drugs approved by a governmental regulatory agency for administration to a patient.

3. The method of claim 1, wherein said test drug is a drug approved by a governmental regulatory agency for administration to a patient.

4. The method of claim 2 or 3, wherein said governmental regulatory agency is the United States Food and Drug Administration.

5. The method of claim 1, wherein said test drug is a drug that is not approved by a governmental regulatory agency.

6. The method of claim 1, wherein said combination exhibits an activity in the assay that is increased relative to the activity of either drug of the combination by itself.

7. The method of claim 1, wherein said combination exhibits an activity in the assay that is decreased relative to the activity of either drug of the combination by itself.

8. A method for screening two-drug or higher order combinations for biological activity, said method comprising the steps of:
(a) creating an array of at least 200 different two-drug or higher order combinations;
(b) testing at least 200 of said combinations in an assay under conditions that ensure that each drug combination/assay contacting is segregated from the others;
(c) recording the result of said testing step (b); and
(d) identifying combinations of drugs that produce a result in said assay that is different from the results produced by any drug of the combination by itself, wherein each identified combinations indicates an interaction among said drugs of said combination.

9. The method of claim 8, wherein at least 200 of said drugs of step (a) are drugs approved by a governmental regulatory agency for administration to a patient.

10. The method of claim 9, wherein said governmental regulatory agency is the United States Food and Drug Administration.

11. The method of claim 8, wherein said combination exhibits an activity in the assay that is increased relative to the activity of any of the drugs of the combination by itself.

12. The method of claim 8, wherein said combination exhibits an activity in the assay that is decreased relative to the activity of any of the drugs of the combination by itself.

13. The method of claim 1 or 10, wherein said library comprises at least 500 different drugs.

14. The method of claim 13, wherein said library comprises at least 1000 drugs.

15. The method of claim 1 or 8, wherein said method is repeated at least three times, using at least three different assays.

16. The method of claim 1 or 8, wherein said method is repeated at least ten times, using at least ten different assays.

17. A method for identifying an interaction between two drugs co-prescribed to a patient, said method comprising the steps of:
(a) providing (i) a test drug; (ii) a drug library comprising drugs that are co-prescribed; and (iii) an assay;
(b) contacting the test drug and at least some of the drugs in the library in the assay under conditions that ensure that each test drug/library drug contacting is segregated from the others;
(c) recording the result of the contacting of the test drug and the library drug in the assay; and
(d) identifying test drug/library drug combinations that produce a result in the assay that is different from the results produced by either drug of the combination by itself.

18. The method of claim 17, wherein said test drug is a drug that is co-prescribed with at least one library drug.

19. The method of claim 17, wherein said test drug is a drug not approved by a governmental regulatory agency.

20. The method of claim 17, wherein said library comprises at least three different drugs.

21. The method of claim 20, wherein said library comprises at least ten different drugs.

22. The method of claim 21, wherein said library comprises at least twenty-five different drugs.

23. The method of claim 17, wherein said combination exhibits an activity in the assay that is increased relative to the activity of either drug of the combination by itself.

24. The method of claim 17, wherein said combination exhibits an activity in the assay that is decreased relative to the activity of either drug of the combination by itself.

25. The method of claim 1 or 17, wherein said providing step (a) is under the control of a client, and each of said contacting step (b), recording step (c) and identifying step (d) is under control of a service provider.

26. The method of claim 1, 8, or 17, wherein said assay comprises one or more living human or non-human cells.

27. The method of claim 26, wherein said cells are cancer cells, immune cells, neurons, or fibroblasts.

28. The method of claim 1, 8, or 17, wherein said assay employs a cell-free system.

29. The method of claim 1, 8, or 17, wherein said assay is an assay that measures toxicity of said drug combinations.

30. The method of claim 1, 8, or 17, wherein said method employs at least one of the following assays: a cytoblot assay; a reporter gene assay; and a fluorescence resonance energy transfer assay.

31. The method of claim 1, 8, or 17, wherein said assay comprises a fluorescent calcium-binding indicator dye, fluorescence microscopy, or gene expression profiling.

32. A library screening system for determining whether a test drug interacts with a member of a drug library in an assay, said system comprising:
(i) a test drug;
(ii) a drug library comprising at least 200 drugs approved by a governmental regulatory agency;
(iii) means for contacting said test drug and at least 200 of the drugs from said drug library in an assay under conditions that ensure that each test drug/library drug contacting is segregated from the others;
(iv) means for recording the result of the contacting; and
(v) means for identifying test drug/library drug combinations that produce a result that is different from the result of either drug of the combination by itself.

33. A library screening system for determining whether a test drug interacts with a member of a drug library in an assay, said system comprising:
(i) a test drug;
(ii) a drug library comprising at least 200 drugs;
(iii) means for contacting said test drug and at least 200 of the drugs from said drug library in an assay under conditions that ensure that each test drug/library drug contacting is segregated from the others;
(iv) means for recording the result of the contacting; and
(v) means for identifying test drug/library drug combinations that produce a result that is different from the result of either drug of the combination by itself.
